(19) United States
(12) Patent Application Publication
(10) Pub. No.: US 2024/0252535 A1
(43) Pub. Date: Aug. 1, 2024

(54) CELLULAR COMPOSITION FOR TREATMENT OF DISEASES, DISORDERS OR CONDITIONS AND METHOD OF USE

(71) Applicant: YZTHERAPEUTIC PERFORMANCE LTD., Rehovot (IL)

(72) Inventor: Yaacov GUY, Rehovot (IL)

(21) Appl. No.: 18/044,120

(22) PCT Filed: Aug. 31, 2021
(86) PCT No.: PCT/IL2021/051064

§ 371 Date: Mar. 6, 2023

Related U.S. Application Data
(60) Provisional application No. 63/073,792, filed on Sep. 2, 2020.

(51) Int. Cl. A61K 35/17; C12N 5/0783; C12N 9/02

(52) U.S. Cl. CPC A61K 35/17; C12N 5/0636; C12N 9/0069; C12N 2501/999; C12Y 113/11017

(57) ABSTRACT

The present invention relates to a cellular composition, more particularly a composition comprising a serotonin receptor-expressing cells that have been exposed to a serotonin receptor agonist and consequently stimulated, for use in the treatment of a disease, disorder or condition; a method of use; and a method for preparing such a composition.

### TECHNICAL FIELD

The present invention relates to a cellular composition, more particularly a composition comprising cells expressing a serotonin (5-HT) receptor that have been exposed to a serotonin receptor agonist and consequently stimulated, for use in the treatment of diseases, disorders or conditions, and to a method of use.

### BACKGROUND ART

In his 2015 review, Szabo (2015) noted that several neurotransmitter receptors involved in the pharmacology of psychedelics, such as serotonin and sigma-1 receptors, also play crucial roles in numerous immunological processes. This field offers promising treatment modalities in the therapy of various diseases including autoimmune and chronic inflammatory conditions, infections, and cancer. “However, the scarcity of available review literature renders the topic unclear and obscure, mostly posing psychedelics as illicit drugs of abuse and not as physiologically relevant molecules or as possible agents of future pharmacotherapies”. Christensen et al. (2016) found that administration of serotonin or sertraline, a selective serotonin reuptake inhibitor (SSRI) actually decreased time to disease progression of a murine model of ovarian cancer and resulted in increased tumor weight. More recently, Santos et al. (2019) discussed the potential usefulness of serotonin receptor drugs for cancer, yet they did not mention the possibility of anti-cancer activities.

It is well-established that cells of the immune system have receptors which bind serotonin (AKA 5-hydroxytryptamine, 5-HT) and compounds referred to as serotonin receptor agonists. The subsequent serotonin receptor effects have each been related to specific 5-HT receptor subsets, including 5-HT_{1A}, 5-HT_{1B}, 5-HT₃, 5-HT_{3A}, 5-HT₇, and 5-HT_{2A}. While having structural differences, all of these receptors bind 5-HT. As such, compounds referred to as serotonin receptor agonists bind to subsets of 5-HT receptors with varying affinity. The affinities of some of these compounds vary so greatly that their binding to some 5-HT subsets is considered insignificant or even nonexistent. With regard to cells of the immune system, Table 1 summarizes the current knowledge regarding the association of 5-HT receptor subsets. The biological effects related to 5-HT receptors depend on the type of cell on which they are expressed and interactions with other cells. These effects include T cell proliferation, secretion of proinflammatory cytokines such as IL-2 and IFN-γ, and activation of the ERK-1-2/NF-κB pathway.

TABLE 1
5-HT subsets associated on human immune cells*

| Cells | 5-HT Substes |
|---|---|
| T cells | 5-HT_{1A}, 5-HT_{1B}, 5-HT_{2A}, 5-HT_{3A}, 5-HT₇ |
| B cells | 5-HT_{1A}, 5-HT_{2A}, 5-HT_{3A} |
| Macrophages | 5-HT_{1A}, 5-HT_{2A}, 5-HT_{2B}, 5-HT_{2C}, 5-HT₇ |
| Monocytes | 5-HT_{1E}, 5-HT_{2A}, 5-HT₄, 5-HT₇ |
| Eosinophils | 5-HT_{1A}, 5-HT_{1B}, 5-HT_{1E}, 5-HT_{2A} 5-HT₆ |
| Neutrophils | 5-HT₇ |
| NK cells | 5-HT_{1A} |
| Mast cells | 5-HT_{1A}, 5-HT_{1B}, 5-HT_{1E}, 5-HT_{2A}, 5-HT_{2B}, |
| | 5-HT_{2C}, 5-HT₃, 5-HT₄, 5-HT₆, 5-HT₇ |
| Dendritic cells | 5-HT_{1B}, 5-HT_{1E}, 5-HT_{2B}, 5-HT₃, 5-HT₄, 5-HT₇ |

*Adapted from Arreola et al., 2015; and Shajib and Khan, 2014

Oxidative stress inflicted by reactive oxygen species (ROS) is assumed to contribute to immunosuppression in the vicinity of a tumor by inhibiting functions of NK cells and other relevant lymphocytes to protect against neoplastic cells (Betten et al., 2001). Early in vitro studies revealed that 5-HT can activate human NK cells by regulating an interaction between NK cells and monocytes (Hellstrand and Hermodsson, 1987 and 1990; Hellstrand et al., 1993), but the mechanistic details of these activating properties are not known. The results of subsequent studies indicate that 5-HT protects NK cells from monocyte-derived inhibitory and apoptosis-inducing signals conveyed by ROS. In the presence of 5-HT, NK cells remain viable and functionally active and can be activated by IL-2 despite the presence of suppressive monocytes.

The availability of 5-HT is regulated by its synthesis, metabolism, secretion from neurons, and uptake into neurons. Indoleamine 2,3-dioxygenase (IDO) is an enzyme that converts the 5-HT precursor, tryptophan (Trp) to kynurenine (Kyn), and thereby limits the amount of Trp available for producing 5-HT (FIG. 1). IDO can also metabolize 5-HT, though monoamine oxidase (MAO) produces the major metabolite of 5-HT, 5-hydroxyindoleacetic acid (5-HIAA) (Hayaishi, 1976). In either case, elevated levels of IDO activity can deplete available 5-HT, which significantly impacts the immune activities.

IDO was found to mediate immune responses, especially those of T lymphocytes (Munn and Mellor, 2013). “IDO contributes to maternal tolerance to semi-allogeneic fetal tissues and transplanted organs, inhibits local tissue inflammation and autoimmunity, and suppresses immunity to cancer and chronic infections. A common theme in these diverse immunologic settings is that IDO contributes to immune regulation via local metabolic changes in the immediate microenvironment and local tissue milieu, and these local changes may ultimately impact the development of systemic immune tolerance.” Ninomiya et al. (2015) demonstrated that IDO expression on tumor cells can also dramatically reduce in vivo CAR-T cell control of CD19⁺ IDO-expressing tumor growth progression in model systems. Their data also indicate that Kyn and hydroxyanthranilic acid may have a role in CAR-T activity suppression.

Kyn itself was found to be immunosuppressive. Also, Kyn metabolites can cause apoptosis, proliferation of Treg and Th17 cells, and deviation of the Th1/Th2 response. Additionally, by directing Trp catabolism to form Kyn, IDO activity reduces the amount of Trp available for producing 5-HT via an alternative metabolic pathway. Wang et al. (2016) summarized IDO's effects on tumor immunity, stating, Host DCs expressing immunosuppressive IDO are found in tumor-draining lymph nodes, and IDO can also be expressed by tumor cells themselves (Munn, 2006). “Most tumors express IDO (Zadori et al., 2016) and IDO can contribute to tumor-induced immunosuppression by starving natural killer (NK)/T cells, which are sensitive to tryptophan deficiency (Uyttenhove et al., 2003; Munn et al., 1998; Della Chiesa et al., 2006; Nonaka et al., 2011).” Christensen et al. (2016) found that increasing 5-HT, either by administering 5-HT or the 5-HT reuptake inhibitor (SSRI), sertraline, to tumor-bearing mice, led to increased tumor weight.

U.S. Pat. Nos. 9,931,347 and 10,336,731 suggested that IDO and/or tryptophan-2,3-dioxygenase (TDO) inhibitors are useful for treating diseases including cancer, infectious diseases, CNS disorders, sepsis-induced hypotension, and other diseases when administered as the sole active component or together with CAR-T cells.

Serotonin receptor agonists like those of Table 2 have potential therapeutic uses for conditions and diseases including autoimmune and chronic inflammatory conditions, infections, and cancer. For example, LSD and psilocybin are known for their anti-depressive effects and may help depression in cancer patients. However, their hallucinogenic effects are most well-known and resulted in their classification as class I controlled substances in the 1960s. Therefore, treating cancer patients with these serotonin agonists is currently not a viable option. Determining ways in which these drugs can be incorporated into therapeutic use would be beneficial for patients with diseases considered as unmet need or which require improved therapies.

TABLE 2
Select serotonin receptor agonists and their reported main specificity

| | Reported | |
|---|---|---|
| Compound | Specificity | Source |
| urapidil | 5-HT_{1A} | van Zwieten et al., 1990 |
| 5-methyl-urapidil | 5-HT_{1A} | Mandal et al., 1991 |
| quipazine | 5-HT_{1A/3} | Maguire et al., 2013 |
| lysergic acid diethylamide | 5-HT₂ | Maguire et al., 2013 |
| (LSD) | | |
| 1-(2,5-dimethoxy-4- | 5-HT_{2A} | Maguire et al., 2013 |
| methylphenyl)-2- | | |
| aminopropane (DOM) | | |
| aripiprazole | 5-HT_{1A} | Citrome, 2015 |
| sumatriptan | 5-HT_{1B/1D} | Rodrigues et al., 2008 |
| CGS 12066B | 5-HT_{1B} | Neale et al., 1987 |
| CP-94,253 | 5-HT_{1B} | Lee and Simansky, 1997 |
| 5-carboxamidotryptamine | 5-HT_{1,7} | Delesalle et al., 2006 |
| (5-CT) | | |
| flesinoxan | 5-HT_{1A} | van Zwieten et al., 1990 |
| mirtazapine | 5-HT_{2A/3} | Fang et al., 2012 |
| m-chlorophenylpiperazine | 5-HT_{2B/2C} | Mazzola-Pomietto |
| (m-CPP) | | et al., 1996 |
| norfenfluramine | 5-HT_{2A/2B} | Wang et al., 2013 |
| ergotamine | 5-HT_{2B} | Silberstein, 1997 |
| liseride | 5-HT_{1A/2A/2B} | Newman-Tancredi |
| | | et al., 2002 |
| fenfluramine | 5-HT_{2C} | |
| dihydroergotamine (DHE) | 5-HT_{1B/1D} | Khalife et al., 2019 |
| pergolide (PER) | 5-HT_{1A/1B/2A/2B/2C} | Newman-Tancredi |
| | | et al., 2002 |
| cabergoline (CAB) | 5-HT_{1A/2A/2B/2C} | Newman-Tancredi |
| | | et al., 2002 |
| terguride | 5-HT_{1A/1D} | Newman-Tancredi |
| | | et al., 2002 |
| piribedil | 5-HT_{1A} | Newman-Tancredi |
| | | et al., 2002 |
| 5-hydroxy-N,N- | 5-HT_{2A/3} | Shen et al., 2010 |
| dimethyltryptamine | | |
| (bufotenine) | | |
| 2-methyl-5-HT | 5-HT₃ | Wang et al., 1994 |
| phenylbiguanide | 5-HT₃ | Tuladhar et al., 2000 |
| 2,5-dimethoxy-4- | 5-HT_{(2A/2C)} | Canal et al., 2012 |
| iodoamphetamine (DOI) | | |
| 3,4-methylenedioxy- | 5-HT_{2B} | |
| methamphetamine (MDMA) | | |
| fluoxetine | 5-HT_{2B} | Hertz et al., 2015 |
| 5-methoxytryptamine | 5-HT_{2A/2B/2C/3} | Toro-Sazo et al., 2019 |
| α-methylserotonin | 5-HT_{2A/2C} | De Almeida et al., 2006 |
| tandospirone | 5-HT₁ | Jin et al., 2019 |
| mosapride | 5-HT₄ | Jin et al., 2019 |
| cisapride | 5-HT₄ | Jin et al., 2019) |
| R-137696 | 5-HT_{1A} | Jin et al., 2019 |
| tegaserod | 5-HT_{4A} | Jin et al., 2019 |
| psilocin | 5-HT_{2A/2B/2C/5/6/7} | Madsen et al., 2019; |
| | | Geiger et al., 2018 |
| psilocybin | 5-HT_{2A/2B/2C/5/6/7} | Madsen et al., 2019; |
| | | Geiger et al., 2018 |
| 8-hydroxy-2-(di-n- | 5-HT_{1A} | van Zwieten et al., 1990 |
| propylamino)tetrarin | | |
| (8-OH-DPAT) | | |
| BW723C86 | 5-HT_{2A-C} | Sharif, 2010 |
| 4-(4-[4-(2-pyrimidinyl)- | 5-HT_{1A} | Okada et al., 1993 |
| piperazin-1-yl]butyl)-2,3,4,5- | | |
| tetrahydro-1,4-benzoxazepine- | | |
| 3,5-dione (SUN8399) | | |
| N,N-dimethyltryptamine | 5-HT_{1A/2A/2C} | Dos Santos et al., 2016 |
| (DMT) | | |
| gepirone | 5-HT_{1A} | Cowen et al., 1990 |
| ipsapirone | 5-HT_{1A} | Cowen et al., 1990 |
| tandospirone | 5-HT_{1A} | Kim, 2012 |
| N-benzylated analogues | 5-HT_{2A} | Nichols et al., 2014 |
| of 2,5-dimethoxy- | | |
| 4-iodophenethylamine | | |
| buspirone | 5-HT_{1A} | Delesalle et al., 2006 |
| (+)-cis-8-hydroxy- | 5-HT_{1A} | Hellstrand and |
| 1-methyl-2-(di-n- | | Hermodsson, 1990 |
| propylamino) tetralin (ALK) | | |
| brexpiprazole | 5-HT_{1A} | Suzuki et al., 2019 |

Bone marrow cells, stem cells, and more recently, chimeric antigen receptor T (CAR-T) lymphocyte cells, following ex vivo manipulations, have been administered to patients for treating malignant cell growth. While promising, these therapies have their limitations. One limitation is their efficacy for treating IDO-positive tumors. Ninomiya et al., (2015) observed that CD19-CARTs inhibited IDO-negative tumor growth but had no effect on IDO-positive tumors. They also observed that tryptophan metabolites inhibited interleukin (IL)-2-, IL-7-, and IL-15-dependent expansion of CAR-T cells; diminished their proliferation, cytotoxicity, and cytokine secretion in vitro in response to CD19 recognition; and increased their apoptosis. Their conclusion was, “because tumor IDO inhibits CD19-CAR-Ts, antagonizing this enzyme may benefit CD19-CAR-T therapy”.

As disclosed in U.S. Pat. No. 10,358,629, the significant potential to effectively treat many diseases with stem cells is widely recognized. Stem cells have been identified in most organs and tissues, and can be found in adult animals and humans. Committed adult stem cells (also referred to as somatic stem cells) were identified long ago in bone marrow. Hematopoietic stem cells (HSCs) are the most well-characterized type of stem cell. These cells, which originate in bone marrow, peripheral blood, cord blood, the fetal liver, and the yolk sac, generate blood cells and give rise to multiple hematopoietic lineages. Stem cells are applied in a form of cellular therapy for local tissue repair and regeneration. These treatments aim to treat disorders in practically all tissues and organs, such as the bladder, intestine, kidney, trachea, eye, heart valves, and bones. In some applications, core cell population (CCP)-derived progenitor cells (which arm at least 1% of which are both CD34 positive and CD45 negative) are used as a therapeutic cell product for, e.g., cancer therapy, tissue regeneration, tissue engineering, and/or tissue replacement. For some applications, desired progenitor cells are prepared from CCP, which have been cultured with proliferation-differentiation-enhancing agents such as cytokines, hormones, and neurotransmitters.

STEMCELL Technologies is an example of a company that commercializes media and media supplements for growing and expanding inter alia immune, epithelial, hematopoietic, kidney, hepatic, and neuronal cells. Media available for T cell expansion include ImmunoCult™-XF T Cell Expansion Medium (serum-free and xeno-free medium for the expansion of human T cells).

As disclosed in US 20200017825, approaches to solving the foregoing problems associated with the culture of single mammalian stem cells have involved complex media formulations comprising an array of small molecule inhibitors. Such media formulations are inadequate, on account of the cost of manufacture and their inefficiency. Accordingly, there remains a need for culture media and methods to enhance the survival and/or proliferation of mammalian stem cells in in vitro cultures. The patents of this company and others concerning medium for CAR-T do not relate to serotonin receptor agonists.

US 20180228866 actually recommends treating T cell preparations, like CAR-T, with a serotonin inhibitor-containing medium to reduce cytokine release syndrome.

### SUMMARY OF INVENTION

In one aspect, the present invention relates to a composition comprising a serotonin (5-HT) receptor-expressing cells, such as bone marrow cells, stem cells, lymphocytes, white blood cells, CAR-T cells, CAR-NK cells, and natural killer cells, for use in the treatment of a disease, disorder or condition, wherein said cells have been exposed to (i) a serotonin receptor agonist; or (ii) a prodrug of said serotonin receptor agonist in the presence of an enzyme capable of converting said prodrug to said serotonin receptor agonist. Examples of diseases, disorders or conditions treatable by this composition include immune-related diseases, disorders or conditions; cardio-related diseases, disorders or conditions; hyperproliferative disorders; and cancer.

In another aspect, the present invention relates to a method of treatment of a disease, disorder or condition, e.g., an immune-related disease, disorder or condition; cardio-related disease, disorder or condition; hyperproliferative disorder; or cancer, in a subject in need thereof, said method comprising the steps of:
(i) treating a composition comprising a serotonin receptor-expressing cells with a serotonin receptor agonist; or a prodrug of said serotonin receptor agonist in the presence of an enzyme capable of converting said prodrug to said serotonin receptor agonist, e.g., by exposing or contacting said cells to said serotonin receptor agonist or prodrug thereof, thereby stimulating said cells; and
(ii) administering a therapeutically effective amount of the stimulated cells obtained in step (i) to said subject to thereby treat said disease, disorder or condition.

The method disclosed herein may further comprise the steps of removing excess, i.e., unbound molecules, of said serotonin receptor agonist or prodrug thereof from said composition, e.g., by washing the stimulated cells obtained in step (i); and optionally diluting the composition thus obtained, prior to step (ii).

In a further aspect, the present invention provides a method for stimulating serotonin receptor-expressing cells, said method comprising contacting said cells with (i) a serotonin receptor agonist; or (ii) a prodrug of said serotonin receptor agonist in the presence of an enzyme capable of converting said prodrug to said serotonin receptor agonist. The stimulated cells obtained in vitro by this method may then be used as a therapeutic product, i.e., administered to a subject in need thereof, to thereby treat a disease, disorder or condition, e.g., an immune-related disease, disorder or condition; cardio-related disease, disorder or condition; hyperproliferative disorder; or cancer.

Accordingly, in yet further aspects, the present invention relates to a composition comprising (i) a serotonin receptor agonist; or (ii) a prodrug of said serotonin receptor agonist in the presence of an enzyme capable of converting said prodrug to said serotonin receptor agonist, as well as to use of such a composition, for stimulating serotonin receptor-expressing cells.

### BRIEF DESCRIPTION OF DRAWINGS

FIG. 1 depicts metabolic pathways of tryptophan (adopted from Mellor et al., Front Immunol., 2017, 8, 1360).

FIG. 2 shows killing of Raji cells in terms of luciferase release by unstimulated CAR-T cells (0), or by CAR-T cells subsequent to their stimulation with 5×10⁻⁵, 1×10⁻⁴, or 2×10⁻⁴ M 8-OH-DPAT. Statistical significance between treatment groups is indicated by the asterisks as follows: *p<0.05, *p<0.01, ****p<0.0001.

FIG. 3 shows killing of Raji cells in terms of luciferase release by unstimulated CAR-T cells (0), or by CAR-T cells subsequent to their stimulation with 5×10⁻⁵, 1×10⁻⁴, or 2×10⁻⁴ M pergolide mesylate. Statistical significance between treatment groups is indicated by the asterisks as follows: p<0.05, **p<0.01, ****p<0.0001.

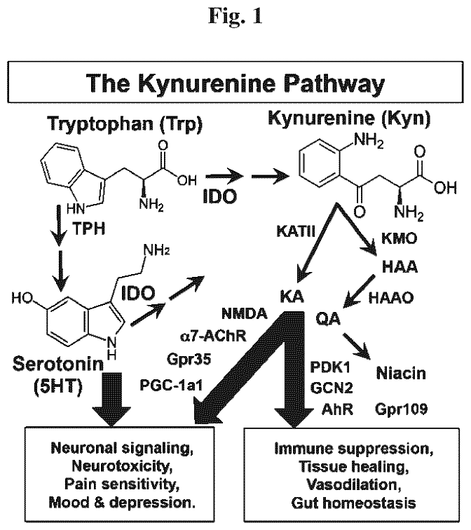

### DETAILED DESCRIPTION

In one aspect, the present invention relates to a composition comprising a 5-HT receptor-expressing cells for use in the treatment of a disease, disorder or condition, i.e., a medical condition, wherein said cells have been exposed to (i) a serotonin receptor agonist; or (ii) a prodrug of said serotonin receptor agonist in the presence of an enzyme capable of converting said prodrug to said serotonin receptor agonist.

The term “receptor agonist”, as used herein, refers to a molecule capable of binding to, or associating with, a specified receptor and consequently activating said receptor to produce a biological response.

The term “serotonin (5-HT) receptor agonist”, as used herein, refers to any molecule capable of binding to, or associating with, one or more of the receptors which bind 5-HT, and consequently activating said receptor to produce a biological response, or to a salt of said molecule.

According to the present invention, cells expressing a 5-HT receptor may express any one of the receptors listed in Table 2, i.e., 5-HT_{1A}, 5-HT_{1B}, 5-HT_{1E}, 5-HT_{2A}, 5-HT_{2B}, 5-HT_{2C}, 5-HT_{3A}, 5-HT₃, 5-HT₄, or 5-HT₇ receptor, as well as any combination of these receptors. Examples of cells expressing a 5-HT receptor include, without limiting, bone marrow cells, stem cells, lymphocytes, white blood cells, CAR-T cells, CAR-NK cells, and natural killer cells.

In certain embodiments, the composition disclosed herein comprises a 5-HT receptor-expressing cells that have been exposed to a serotonin receptor agonist such as a tryptamine (an indolamine metabolite of the essential amino acid tryptophan), phenethylamine, or ergoline, or a derivative, analog, or salt thereof.

The terms “derivative” and “analog” as used herein with respect to a serotonin receptor agonist, more specifically with respect to a tryptamine, phenethylamine, or ergoline, refer to any chemical derivative of said serotonin receptor agonist, having a biological activity identical or similar to that of the corresponding, i.e., non-derivatized, serotonin receptor agonist, i.e., capable of binding to, or associating with, a 5-HT receptor with specificity and selectivity that are either identical or similar to those of the corresponding agonist, and consequently stimulating the cell expressing said receptor.

The term “salt” as used herein with respect to a serotonin receptor agonist, more specifically with respect to a tryptamine, phenethylamine, ergoline, or a derivative or analog thereof, refers to any possible salt of said serotonin receptor agonist including, without being limited to, the hydrochloride, hydrobromide, sulfate, sulfonate, phosphate, carboxylate, acetate, maleate, fumarate, tartarate, citrate, succinate, mesylate, esylate, tosylate, benzenesulfonate, and benzoate salt of said serotonin receptor agonist.

Specific examples of serotonin receptor agonists include urapidil, 5-methyl-urapidil, quipazine, lysergic acid diethylamide (LSD), 1-(2,5-dimethoxy-4-methylphenyl)-2-aminopropane (DOM), CGS 12066B. CP-94,253, flesinoxan, mirtazapine, m-chlorophenylpiperazine, norfenfluramine, ergotamine, methylergonovine, liseride, fenfluramine, dihydroergotamine, pergolide, cabergoline, terguride, piribedil, bufotenine, 2-methyl-5-HT, phenylbiguanide, 2,5-dimethoxy-4-iodoamphetamine, 3,4-methylenedioxy-methamphetamine, fluoxetine, 5-carboxamidotryptamine (5-CT), 5-methoxytryptamine, 5-methoxy-α-methyltryptamine (5-MeO-AMT), N,N-dimethyltryptamine (DMT), 4-fluoro-N,N-dimethyltryptamine, 5-methoxy-N,N-dimcthyltryptamine (5-MeO-DMT), N,N-diisopropyltryptamine (DiPT), 4-hydroxy-N,N-diisopropyltryptamine (4-OH-DiPT), 4-hydroxy-N-methyl-N-ethyltryptamine (4-OH-MET), 5-methoxy-N-methyl-N-isopropyl tryptamine (5-MeO-MiPT), 5-methoxydiisopropyltryptamine (5-MeO-DiPT), α-methylserotonin, tandospirone, psilocin (4-hydroxy-N,N-dimethyltryptamine), 1-methylpsilocin, N-butylpsilocin, 8-hydroxy-2-(di-n-propylamino)tetralin (8-OH-DPAT). BW723C86, 4-(4-[4-(2-pyrimidinyl)piperazin-1-yl]butyl)-2,3,4,5-tetrahydro-1,4-bcnzoxazepine-3,5-dione, gepirone, ipsapirone, tandospirone. N-benzylated analogues of 2,5-dimethoxy-4-iodophenethylamine, buspirone, (+)-cis-8-hydroxy-1-methyl-2-(di-n-propylamino)tetralin, and brexpiprazole, as well as any combination thereof. As shown in Table 2 herein, some of these serotonin receptor agonists are capable of binding to, or associating with, more than one 5-HT receptor.

Examples of analogs of serotonin receptor agonists include, without limiting, the relatively water-soluble aripiprazole monohydrate; as well as dihydropyrano-[3,2-e]indole derivatives of serotonin, such as 1-(2-aminoethyl)-3-methyl-8,9-dihydropyrano[3,2-e]indole (CP-132,484) and 1-(2-aminoethyl)-8,9-dihydropyrano-[3,2-e]indole that have similar or enhanced 5-HT₂ receptor specificity relative to the parent (Macor et al., 1992).

In other embodiments, the composition disclosed herein comprises a 5-HT receptor-expressing cells that have been exposed to a prodrug of a serotonin receptor agonist as described above, in the presence of an enzyme, e.g., a phosphatase such as alkaline phosphatase, an esterase, or a hydrolase, capable of converting said prodrug to said serotonin receptor agonist.

The term “prodrug” as used herein with respect to a serotonin receptor agonist refers to a chemical derivative of said serotonin receptor agonist, e.g., a phosphorylated form thereof, either devoid of serotonergic activity or having an attenuated serotonergic activity compared to the corresponding serotonin receptor agonist, which is converted to its biologically active form upon enzymatic cleavage, e.g., hydrolysis of the phosphate group.

Specific examples of prodrugs of serotonin receptor agonists include psilocybin and N-methylpsilocybin, which are phosphorylated forms of psilocin and 1-methylpsilocin, and are hydrolyzed, e.g., by alkaline phosphatase (ALP, ALKP) capable of removing the phosphate group to generate psilocin and 1-methylpsilocin, respectively; aripiprazole lauroxil, which is a lauric acid ester of N-hydroxymethyl aripiprazole, and is cleaved by an esterase to generate the active N-hydroxymethyl aripiprazole (Citrome, 2015); and acylated forms of LSD such as 1-acetyl-LSD (ALD-52), 1-propionyl-LSD (1P-LSD) and 1-butyryl-LSD (1P-LSD), which are hydrolyzed by a hydrolase to generate the active LSD (Brandt et al., 2019).

In certain particular embodiments, the cells comprised within the composition of the invention have been exposed to the serotonin receptor agonist pergolide or 8-OH-DPAT, or to a salt thereof. In other particular embodiments, the cells comprised within the composition of the invention have been exposed to the serotonin receptor agonist psilocin, or a derivative thereof such as 1-methylpsilocin; or to a prodrug of said agonist such as psilocybin or N-methylpsilocybin, in the presence of alkaline phosphatase capable of hydrolyzing psilocybin and N-methylpsilocybin to psilocin and 1-methylpsilocin, respectively. In a specific embodiment, the cells comprised within the composition have been exposed to psilocybin, in the presence of alkaline phosphatase. As has been shown, psilocybin binds to multiple 5-HT receptors, but it has the highest affinity for 5-HT_{2A} (K₁=6 nM) and to a lesser extent for 5-HT_{1A} receptors (Kᵢ=190 nM) (Geiger et al., 2018).

In certain embodiments, the cells comprised within the composition of the invention have been exposed to a serotonin receptor agonist or a prodrug thereof, each as defined in any one of the embodiments above, at an agonist/prodrug concentration of from about 1 μM to about 1 mM, e.g., at a concentration of from about 10 μM to about 800 μM, from about 20 μM to about 600 μM, from about 40 μM to about 600 μM, from about 50 μM to about 500 μM, or from about 100 μM to about 250 μM.

In certain embodiments, the disease, disorder or condition treated by the composition disclosed herein, according to any one of the embodiments above, is an immune-related disease, disorder or condition; a cardio-related disease, disorder or condition; a hyperproliferative disorder; or cancer.

The term “immune-related disease, disorder or condition” as used herein refers to a disease, disorder, or condition in which significant dysfunction of immune system cells is measurable and can be associated with pathological conditions. Examples of immune-related disease, disorder or condition, without being limited to, include rheumatoid arthritis, osteoporosis, inflammatory bowel disease, ulcerative colitis, Crohn's disease, malaria, and trypanosomiasis.

The term “cardio-related disease, disorder or condition” as used herein refers to a condition affecting the heart or blood vessels that is usually associated with a build-up of fatty deposits inside the arteries (atherosclerosis) and an increased risk of blood clots. Examples of cardio-related disease, disorder or condition include, e.g., coronary heart disease, chronic heart failure, myocardial infarction, and stroke.

The term “hyperproliferative” disorder” as used herein refers to any disease or disorder in which cells proliferate at rates higher than in a non-disease or non-disorder state. Non-cancerous hyperproliferative diseases or disorders include, e.g., psoriasis or benign hyperplasia of the skin or prostate. The term “cancer” as used herein refers to the physiological condition typically characterized by unregulated cell growth.

According to the present invention, the hyperproliferative disorder or cancer treated with the composition disclosed herein may be present at any location in the body, e.g., in the lung, thyroid, head or neck, nasopharynx, throat, nose or sinuses, brain, spine, breast, adrenal gland, pituitary gland, thyroid, lymph, gastrointestinal, mouth, esophagus, stomach, duodenum, ileum, jejunum, small intestine, colon, rectum, genito-urinary tract, uterus, ovary, cervix, endometrial, bladder, testicle, prostate, kidney, pancreas, liver, bone, bone marrow, lymph, blood, skin, or muscle.

In certain embodiments, the disease, disorder or condition treated by the composition disclosed herein is cancer. Examples of cancers treatable by said composition include, without being limited to, a primary solid cancer such as melanoma, renal cell carcinoma, colon cancer, breast cancer, lung cancer, prostate cancer, bladder cancer, brain cancer, adenocarcinoma of the pancreas, and head and neck tumor, or a metastasis thereof; or a hematological malignancy, i.e., a cancer of the blood or bone marrow, such as leukemia and lymphoma.

In another aspect, the present invention relates to a method for treatment of a disease, disorder or condition in a subject in need thereof, said method comprising the steps of: (i) treating a composition comprising a 5-HT receptor-expressing cells as defined above, e.g., bone marrow cells, stem cells, lymphocytes, white blood cells, CAR-T cells, CAR-NK cells, and natural killer cells expressing, e.g., 5-HT_{1A}, 5-HT_{1B}, 5-HT_{1E}, 5-HT_{2A}, 5-HT_{2B}, 5-HT_{2C}, 5-HT_{3A}, 5-HT₃, 5-HT₄, or 5-HT₇ receptors, or a combination thereof, with a serotonin receptor agonist as defined in any one of the embodiments above; or a prodrug of said serotonin receptor agonist as defined in any one of the embodiments above, in the presence of an enzyme capable of converting said prodrug to said serotonin receptor agonist, thereby stimulating said cells; and (ii) administering a therapeutically effective amount of the stimulated cells obtained to said subject to thereby treat said disease, disorder or condition.

In certain embodiments, the method of the present invention further comprises the steps of removing excess, i.e., unbound molecules, of said serotonin receptor agonist and/or prodrug thereof from said composition, e.g., by washing the stimulated cells obtained in step (i); and optionally diluting the composition thus obtained, prior to step (ii).

In certain embodiments, the 5-HT receptor-expressing cells treated in step (i) are autologous cells obtained from the subject to which the stimulated cells obtained are to be administered in step (ii).

In other embodiments, the 5-HT receptor-expressing cells treated in step (i) are allogeneic cells obtained from a donor, i.e., derived from an individual of the same species, other than the one to whom the stimulated cells obtained are to be administered in step (ii).

In particular embodiments, the cells are treated in step (i) with psilocin or a derivative thereof such as 1-methylpsilocin; or with a prodrug of the aforesaid such as psilocybin or N-methylpsilocybin, respectively, in the presence of a phosphatase such as alkaline phosphatase. In a specific embodiment, the cells are exposed to psilocybin, in the presence of alkaline phosphatase capable of hydrolyzing psilocybin to psilocin.

In certain embodiments, step (i) of the method disclosed herein is carried out by exposing said cells to a serotonin receptor agonist as defined herein, e.g., to pergolide, 8-OH-DPAT, psilocin, 1-methylpsilocin, or a salt thereof; or to a prodrug of the aforesaid as defined herein, at an agonist/prodrug concentration of from about 1 μM to about 1 mM, e.g., at a concentration of from about 10 μM to about 800 PM, from about 20 μM to about 600 μM, from about 40 μM to about 600 μM, from about 50 μM to about 500 μM, or from about 100 μM to about 250 μM.

The term “subject” as used herein refers to any mammal, e.g., a human, non-human primate, horse, ferret, dog, cat, cow, and goat. In a preferred embodiment, the term “subject” denotes a human, i.e., an individual.

The term “treatment” as used herein refers to the administering of a therapeutic amount of a composition as defined above, i.e., a composition comprising a 5-HT receptor-expressing cells, e.g., bone marrow cells, stem cells, lymphocytes, white blood cells, CAR-T cells, CAR-NK cells, and natural killer cells, which have been exposed to a serotonin receptor agonist as defined above and are thus highly stimulated, to enable effective responses such as cytokine production and cytolytic activity of abnormal or foreign cells, to thereby ameliorate undesired symptoms associated with said medical condition; slow down the progression of said medical condition; slow down the deterioration of symptoms; enhance the onset of remission period; slow down the irreversible damage caused in the progressive chronic stage of said medical condition; delay the onset of said progressive stage; lessen the severity or cure said medical condition; improve survival rate and/or more rapid recovery.

In cases the medical condition treated according to the method of the invention is cancer, the term “treatment” as used herein refers to promotion of anticancer activity involving activities such as modification of cytokine production and secretion and cytolytic activity.

The term “therapeutically effective amount” as used herein means an amount of said stimulated cells that will promote the biological or medical response that is being sought. The amount must be effective to improve a subject's health as described above. The effective amount is typically determined in appropriately designed clinical trials (dose range studies) and the person versed in the art will know how to properly conduct such trials to determine the effective amount.

In certain embodiments, the disease, disorder or condition treated by the method of the present invention, according to any one of the embodiments above, is an immune-related disease, disorder or condition; a cardio-related disease, disorder or condition; a hyperproliferative disorder; or cancer, each as defined above.

In a further aspect, the present invention provides a method for stimulating serotonin receptor-expressing cells, said method comprising contacting said cells with (i) a serotonin receptor agonist; or (ii) a prodrug of said serotonin receptor agonist in the presence of an enzyme capable of converting said prodrug to said serotonin receptor agonist. The stimulated cells obtained in vitro by this method may then be used as a therapeutic product, or for making a therapeutic product, e.g., by administering to a subject in need thereof, to thereby treat a disease, disorder or condition, e.g., an immune-related disease, disorder or condition; cardio-related disease, disorder or condition; hyperproliferative disorder; or cancer.

Accordingly, in yet further aspects, the present invention relates to a composition comprising (i) a serotonin receptor agonist; or (ii) a prodrug of said serotonin receptor agonist in the presence of an enzyme capable of converting said prodrug to said serotonin receptor agonist, as well as to use of such a composition, for stimulating serotonin receptor-expressing cells, which may then be used as a therapeutic product, or for making a therapeutic product.

Unless otherwise indicated, all numbers expressing, e.g., the concentration of a serotonin receptor agonist or prodrug thereof used to treat the 5-HT receptor-expressing cells, used in this specification are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Example 1. Preparation of Serotonergic Agonist Media and Treatment of CAR-T Cells

Pergolide mesylate and 8-OH-DPAT (Sigma) were each dissolved in dimethyl sulfoxide (DMSO) to 2×10⁻² M. The solutions were subsequently diluted in Basal CAR-T medium (ProMab (Richmond, CA)) supplemented with 10% fetal bovine serum, 100U/mL sodium penicillin G (Rafa, Jerusalem) and 1000U/mL streptomycin (Rafa, Jerusalem), in a serial manner, to the indicated final concentrations. CD19 scFv-4-1BB-CD3ζ CAR-T cells (CAT. PM-CAR1002-1M) purchased from ProMab (Richmond, CA) were incubated with the diluted pergolide mesylate or 8-OH-DPAT for one hour, at 37° C. and 5% C02. The cells were pelleted by centrifugation and washed with regular cell growth medium to remove pergolide mesylate and 8-OH-DPAT prior to co-culture with target luciferase Raji cells.

### Example 2. Assessment of CAR-T Cell Killing of Luciferase-Expressing Raji Cells

Luciferase Raji cells stably expressing luciferase (Raji/NF-kB Reporter (Luc) stable cell line Cat. CL-1280) were purchased from FenicsBio (Halethorpe, MD) and grown in RPMI medium (Gibco Cat. 21875-034) supplemented with 10% fetal bovine serum, 100U/mL sodium penicillin G (Rafa, Jerusalem) and 1000 U/mL streptomycin (Rafa, Jerusalem), at 37° C. and 5% C02.

Target 2.5×10³ Raji-luc cells were cultured, at 37° C. and 5% CO₂, in each well of 96-well plates, either in medium, medium containing 1% Triton-X100 (Sigma), or with effector CAR-T cells, at an effector:target cell ratio of 0.5:1, 1:1, or 3:1 for 4 hours. Four replicate wells were prepared for each type of sample. Following the incubation period, the cells were incubated with 50 μL of reconstituted reagent (Bright-Glo Luciferase Assay System, E2620) for 2 min to detect the fluorescence intensity (FLUOstar OMEGA). The fluorescence intensity of the 1% Triton-X100 control was set as 100% killing.

### Example 3. In Vitro Killing of Raji Cells Depends on the Serotonergic Agonist Concentration During Pretreatment of CAR-T Cells

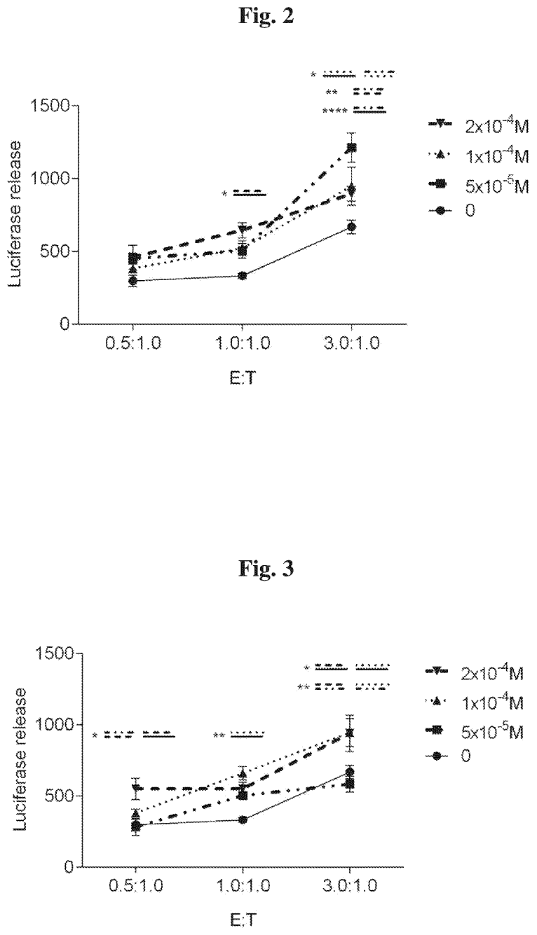

Exposing 2500 Raji cells to 1% Triton-X100 resulted in release of approximately 1000 fluorescence units. As shown in FIG. 2 and FIG. 3, co-culture of the Raji cells with CAR-T cells, at effector:target (E:T) cell ratios of 0.5:1, 1:1, or 3:1, led to release of luciferase activity that increased from 36% to 71% with increasing cell ratio. As shown in FIG. 2, preincubation of CAR-T cells, in the presence of each concentration of 8-OH-DPAT, resulted in enhanced killing of Raji cells. Killing was enhanced 2-3-fold such that the level of killing observed, when the E:T ratio was 0.5:1, equaled or exceeded the level killing observed when the E:T ratio with unstimulated CAR-T cells was 1:1. The level of killing observed, when the E:T ratio was 1:1 with 8-OH-DPAT stimulated CAR-T cells, approximately equaled the level killing of observed when the E:T ratio with unstimulated CAR-T cells was 3:1. Following 8-OH-DPAT stimulation of CAR-T cells, the level of killing attained was approximately 100% when the E:T ratio was 3:1.

As shown in FIG. 3, preincubation of CAR-T cells, in the presence of each concentration of pergolide mesylate, resulted in enhanced killing of Raji cells. Killing was enhanced 2-3-fold such that the level of killing observed, when the E:T ratio was 0.5:1, equaled or exceeded the level killing of observed when the E:T ratio with unstimulated CAR-T cells was 1:1. The level of killing observed, when the E:T ratio was 1:1 with pergolide mesylate stimulated CAR-T cells, approximately equaled the level killing of observed when the E:T ratio with unstimulated CAR-T cells was 3:1. Following pergolide mesylate stimulation of CAR-T cells, the level of killing attained was approximately 100% when the E:T ratio was 3:1. Therefore, pretreatment of CAR-T cells with serotonergic agonists is a means for enhancing the efficacy of CAR-T cells for treating cancer.

### Example 4. In Vivo Killing of Raji Cells by Serotonergic Agonist Pretreated CAR-T Cells

Fifty 6-8-week-old NOD/SCID/γ-chain^{-/-} (NSG) mice (Stock #5557; from The Jackson Laboratory), are acclimated for 7 days and maintained under pathogen-free conditions. The study protocol is approved by the Israeli National Animal Care and Use Committee.

### Example 5. Xenograft Model of Human Burkitt Lymphoma

The mammalian expression construct stable clone of firefly luciferase-expressing (Raji/NF-kB Reporter (Luc) stable cell line Cat. CL-1280) was purchased from FenicsBio (Halethorpe, MD). These cells (1×10⁶ cells/0.2 mL) are subcutaneously injected into the NSG mice (NOD.Cg-PRkdc^{scid}Il2rg^{tmWj1}/SzJ; 6 weeks; The Jackson Laboratory). Tumor engraftment and progression are evaluated using a caliper. When the average tumor size reaches 50 mm³ for about 50% of the experimental animals, treatments according to Table 3 below will be initiated. After tumor engraftment is verified, freshly prepared 2×10⁶ CAR-T cells or buffer are intravenously injected into each mouse once. The control group receives an identical amount of PBS, alone.

An animal for which the tumor size reaches 2000 mm³ is removed from the study and terminated in the same manner as mice which survive the study. After up to 4 weeks of monitoring, all of the animals are terminated by exsanguination from the heart under full anesthesia with CO₂. The tumors are removed, weighed, and histologically examined.

Tumor progression in xenografted mice is monitored weekly by tumor volume measurement and by in vivo bioluminescent imaging (Chu, et al. 2015). Tumor progression and mouse survival are monitored until death, after sacrifice when the tumor size reaches or exceeds 2 cm³, or at the end of the 4-week study period.

TABLE 3

| Treatment group | n | CAR-T cells/mouse |
|---|---|---|
| buffer | 10 | 2 × 10⁶ |
| 10⁻⁴M 8-OH-DPAT:1 | 10 | 10⁶ |
| 2 × 10⁵M 8-OH-DPAT:2 | 10 | 2 × 10⁶ |
| 10⁻⁴M pergolide mesylate:1 | 10 | 10⁶ |
| 2 × 10⁴M pergolide mesylate:2 | 10 | 2 × 10⁶ |

### REFERENCES

Arreola R., Becerril-Villanueva E., Cruz-Fuentes C., Velasco-Velazquez M.A., Garces-Alvarez M.E., Hurtado-Alvarado G., Quintero-Fabian S., Pavon L., Immunomodulatory effects mediated by serotonin. J Immunol Res., 2015, 354957
Betten A., Dahlgren C., Hermodsson S., Hellstrand K., Serotonin protects NK cells against oxidatively induced functional inhibition and apoptosis. J. Leukocyte Biol., 2001, 70, 65-72
Brandt S. D., Kavanagh P. V., Westphal F., Stratford A., Elliott S. P., Dowlingb G., Wallach J., Halberstadt A. L., Return of the lysergamides. Part V: Analytical and behavioural characterization of 1-butanoyl-d-lysergic acid diethylamide (1BLSD). Drug Test Anal., 2019, 11, 1122-1133
Canal C. E., Morgan D., Head-twitch response in rodents induced by the hallucinogen 2,5-dimethoxy-4-iodoamphetamine: a comprehensive history, a re-evaluation of mechanisms, and its utility as a model. Drug Test Anal., 2012, 4(7-8), 556-76
Christensen D. K., Armaiz-Pena G. N., Ramirez E., Matsuo K., Zimmerman B., Zand B., Shinn E., Goodheart M. J., Bender D., Thaker P. H., Ahmed A., Penedo F. J., DeGeest K., Mendez L., Domann F., Sood A. K., Lutgendorf S. K., SSRI use and clinical outcomes in epithelial ovarian cancer. Oncotarget, 2016, 7, 33179
Chu Y. Hochberg J. Yahr A. Ayello J, van de Ven C, Barth M, Czuczman M, Cairo M S. Targeting CD20⁺ aggressive B-cell non-Hodgkin lymphoma by anti-CD20 CAR mRNA-modified expanded natural killer cells in vitro and in NSG mice. Cancer Immunol Res. 2015 3, 333-44
Citrome L., Aripiprazole long-acting injectable formulations for schizophrenia: aripiprazole monohydrate and aripiprazole lauroxil. Expert Review of Clinical Pharmacology, 2015, 9(2), 169-186
Cowen P. J., Anderson I. M., Grahame-Smith D. G., Neuroendocrine Effects of Azapirones. Journal of Clinical Psychopharmacology, 1990, 10(Supplement), 21S-25S
de Almeida R. M., Giovenardi M., da Silva S. P., de Oliveira V. P., Stein D. J., The effect of 5-HT (2a/2c) receptor agonist microinjected into central amygdaloid nucleus and median preoptic area on maternal aggressive behavior in rats. Braz J Psychiatry, 2006, 28, 130-4
Delesalle C., Deprez P., Schuurkes J. A. J., Lefebvre A., Contractile effects of 5-hydroxytryptamine and 5-carboxamidotryptamine in the equine jejunum. Br J Pharmacol., 2006, 147, 23-35
Della Chiesa M., Carlomagno S., Frumento G., Balsamo M., Cantoni C., Conte R., Moretta L., Moretta A., Vitale M., The tryptophan catabolite L-kynurenine inhibits the surface expression of NKp46- and NKG2D-activating receptors and regulates NK-cell function. Blood, 2006, 108, 4118-4125
Dos Santos R.G., Osório F. L., Crippa J.A.S., Hallak J. E. C., Classical hallucinogens and neuroimaging: A systematic review of human studies. Neuroscience & Biobehavioral Reviews, 2016, 71, 715-728
Fang C. K., Chen H. W., Chiang I-T., Chen C. C., Liao J., Su T-P., Tung C-Y., Uchitomi Y., Hwang J-J., Mirtazapine inhibits tumor growth via immune response and serotonin receptor system. PLoS ONE, 2012, 7(7), e38886
Geiger H. A., Wurst M. G., Daniels R. N., DARK classics in chemical neuroscience: psilocybin. ACS Chem Neurosci., 2018, 9, 2438-2447
Hayaishi O., Properties and function of indoleamine 2,3-dioxygenase. J Biochem., 1976, 79(4), 13P-21P
Hellstrand K., Hermodsson S., Role of serotonin in the regulation of human natural killer cell cytotoxicity. J. Immunol., 1987, 139, 869-875
Hellstrand K., Hermodsson S., Monocyte-mediated suppression of IL-2-induced NK cell activation. Regulation by 5-HT_{1A}-type serotonin receptors. Scand. J. Immunol., 1990, 32, 183-192
Hellstrand K., Czerkinsky C., Ricksten A., Jansson B., Asea A., Kylefjord H., Hermodsson S., Role of serotonin in the regulation of interferon-gamma production by human natural killer cells. J. Interferon Res., 1993, 13, 33-38
Hertz L., Rothman D. L., Li B., Peng L., Response: commentary: chronic SSRI stimulation of astrocytic 5-HT_{2B} receptors change multiple gene expressions/editings and metabolism of glutamate, glucose and glycogen: a potential paradigm shift. Front Behav Neurosci., 2015, 9, 308
Jin M., Mo Y., Ye K., Chen M., Liu Y., He C., Efficacy of serotonin receptor agonists in the treatment of functional dyspepsia: a meta-analysis. Arch Med Sci., 2019, 15, 23-32
Khalife J., Lauritsen C. G., Liang J., Shah S. O., DHE-induced peripheral arterial vasospasm in primary raynaud phenomenon: case report. Neurohospitalist, 2019, 9, 113-115
Kim K., Neuroimmunological mechanism of pruritus in atopic dermatitis focused on the role of serotonin. Biomol Ther (Seoul)., 2012, 20(6), 506-12
Lee M. D., Simansky K. J., CP-94,253: a selective serotonin1B (5-HT_{1B}) agonist that promotes satiety. Psychopharmacology (Berl)., 1997, 31, 264-70
Macor J. E., Fox C. B., Johnson C., Koe B. K., Lebel L. A., Zorn S. H., 1-(2-Aminoethyl)-3-methyl-8,9-dihydropyrano[3,2-e]indole: a rotationally restricted phenolic analog of the neurotransmitter serotonin and agonist selective for serotonin (5-HT₂-type) receptors. Journal of Medicinal Chemistry, 1992, 35, 3625-3632
Madsen M. K., Fisher P. M., Burmester D., Dyssegaard A., Stenbaek D. S., Kristiansen S., Johansen S. S., Lehel S., Linnet K., Svarer C., Erritzoe D., Ozenne B., Knudsen G. M., Neuropsychopharmacology, 2019, 44, 1328-1334
Maguire D. R., Li J-X., Koek W., France C. P., Effects of 1-(2,5-dimethoxy-4-methylphenyl)-2-aminopropane (DOM) and quipazine on heroin self-administration in rhesus monkeys. Psychopharmacology (Berl)., 2013, 225(1), 173-185
Mandal A. K., Kellar K. J., Gillis R. A., The role of serotonin-1A receptor activation and alpha-1 adrenoceptor blockade in the hypotensive effect of 5-methyl-urapidil. Journal of Pharmacology and Experimental Therapeutics, 1991, 257, 861-869
Mazzola-Pomietto P., Aulakh C. S., Wozniak K. M., Murphy D. L., Evidence thatm-chlorophenylpiperazine-induced hyperthermia in rats is mediated by stimulation of 5-HT2C receptors. Psychopharmacology, 1996, 123(4), 333-339
Munn D. H., Zhou M., Attwood J. T., Bondarev I., Conway S. J., Marshall B., Brown C., Mellor A. L., Prevention of allogeneic fetal rejection by tryptophan catabolism. Science, 1998, 281, 1191-1193
Munn D. H. Indoleamine 2,3-dioxygenase, tumor-induced tolerance and counter-regulation. Curr Opin Immunol. 2006; 18, 220-5.
Munn D. H., Mellor A. L., Indoleamine 2,3 dioxygenase and metabolic control of immune responses. Trends Immunol., 2013, 34, 137-43
Neale R. F., Fallon S. L., Boyar W. C., Wasley J. W., Martin L. L., Stone G. A., Glaeser B. S., Sinton C. M., Williams M., Biochemical and pharmacological characterization of CGS 12066B, a selective serotonin-1B agonist. Eur J Pharmacol., 1987, 136, 1-9
Newman-Tancredi A., Differential Actions of Antiparkinson Agents at Multiple Classes of Monoaminergic Receptor. III. Agonist and Antagonist Properties at Serotonin, 5-HT1 and 5-HT2, Receptor Subtypes. Journal of Pharmacology and Experimental Therapeutics, 2002, 303(2), 815-822
Nichols D. E., Sassano M. F., Halberstadt A. D., Klein L. M., Brandt S. D., Elliott S. P., Fiedler W. J., N-Benzyl-5-methoxytryptamines as potent serotonin 5-HT2 receptor family agonists and comparison with a series of phenethylamine analogues. ACS Chem. Neurosci., 2014, 6, 1165-1175
Ninomiya S., Narala N., Huye L., Yagyu S., Savoldo B., Dotti G., Heslop H. E., Brenner M. K., Rooney C. M., Ramos C. A., Tumor indoleamine 2,3-dioxygenase (IDO) inhibits CD19-CAR T cells and is downregulated by lymphodepleting drugs. Blood, 2015, 125(25), 3905-16
Nonaka H., Saga Y., Fujiwara H., Akimoto H., Yamada A., Kagawa S., Takei Y., Machida S., Takikawa O., Suzuki M., Indoleamine 2,3-dioxygenase promotes peritoneal dissemination of ovarian cancer through inhibition of natural killer cell function and angiogenesis promotion. Int J Oncol., 2011, 38, 113-120
Okada F., Torii Y., Saito H., Matsuki N., Antiemetic effects of serotonergic 5-HT_{1A}-receptor agonists in Suncus murinus. Jpn J Pharmacol., 1993, 64(2), 109-14
Rodrigues T., Moreira R., Guedes R. C., Iley J., Lopes F., Unanticipated acyloxymethylation of sumatriptan indole nitrogen atom and its implications in prodrug design. Archiv Der Pharmazie, 2008, 341(6), 344-350
Santos R. G. D., Bouso J. C., Hallak J. E. C., Serotonergic hallucinogens/psychedelics could be promising treatments for depressive and anxiety disorders in end-stage cancer. BMC Psychiatry, 2019, 19, 321
Shajib M. S., Khan W. I., The role of serotonin and its receptors in activation of immune responses and inflammation. Acta Physiol (Oxf), 2014, 213, 561-574
Sharif A., Serotonin-2 receptor agonists as novel ocular hypotensive agents and their cellular and molecular mechanisms of action: novel drug targets for glaucoma treatment. Current Drug Targets, 2010, 11(8), 978-993
Shen H-W., Jiang X-L., Winter J. C., Yu A-M., Psychedelic 5-methoxy-N,N-dimethyltryptamine: metabolism, pharmacokinetics, drug interactions, and pharmacological actions. Curr Drug Metab., 2010, 11(8), 659-66
Silberstein S. D., The pharmacology of ergotamine and dihydroergotaminde. Headache, 1997, 37(Suppl 1), S15-25
Suzuki S., Yamamoto M., Togashi K., Sanomachi T., Sugai A., Seino S., Yoshioka T., Kitanaka C., Okada M., In vitro and in vivo anti-tumor effects of brexpiprazole, a newly-developed serotonin-dopamine activity modulator with an improved safety profile. Oncotarget, 2019, 10, 3547-3558
Szabo A., Psychedelics and immunomodulation: novel approaches and therapeutic opportunities. Front Immunol., 2015, 6, 358
Toro-Sazo M., Brea J., Loza M. I., Cimadevila M., Cassels B. K., 5-HT₂ receptor binding, functional activity and selectivity in N-benzyltryptamines. PLoS ONE, 2019, 14(1), e0209804
Tuladhar B. R., Womack M. D., Naylor R. J., Pharmacological characterization of the 5-HT receptor-mediated contraction in the mouse isolated ileum. Br J Pharmacol., 2000, 131(8), 1716-1722
Uyttenhove C., Pilotte L., Théate I., Stroobant V., Colau D., Parmentier N., Boon T., Van den Eynde B. J., Evidence for a tumoral immune resistance mechanism based on tryptophan degradation by indoleamine 2,3-dioxygenase. Nat Med, 2003, 9, 1269-1274
van Zwieten P. A., Blauw G. J., van Brummelen P., The role of 5-hydroxytryptamine and 5-hydroxytryptaminergic mechanisms in hypertension. Br J Clin Pharmacol., 1990, 30 Suppl 1(Suppl 1), 69S-74S
Wang R. Y., Ashby Jr C. R., Edwards E., Zhang J. Y., The role of 5-HT3-like receptors in the action of clozapine. J Clin Psychiatry., 1994, 55 Suppl B, 23-6
Wang C., Jiang Y., Ma J., Wu H., Wacker D., Katritch V., Han G. W., Liu W., Huang X-P., Vardy E., McCorvy J. D., Gao X., Zhou X. E., Melcher K, Zhang C., Bai F., Yang H., Yang L., Jiang H., Roth B. L., Cherezov V., Stevens R. C., Xu H. E., Structural basis for molecular recognition at serotonin receptors. Science, 2013, 340(6132), 610-4
Wang D., Saga Y., Sato N., Nakamura T., Takikawa O., Mizukami H., Matsubara S., Fu H., The hepatocyte growth factor antagonist NK4 inhibits indoleamine-2,3-dioxygenase expression via the c-Met-phosphatidylinositol 3-kinase-AKT signaling pathway. Internat. J. Oncol., 2016, 48, 2303-2309
Zadori D., Klivenyi P., Plangar I., Toldi J., Vecsei L., Endogenous neuroprotection in chronic neurodegenerative disorders: with particular regard to the kynurenines. J Cell Mol Med. 2011, 15, 701-17

1-32. (canceled)

33. A method of treatment of a disease, disorder or condition in a subject in need thereof said method comprising the steps of:
(i) contacting a composition comprising serotonin (5-HT) receptor-expressing cells with a serotonin receptor agonist; or a prodrug of said serotonin receptor agonist in the presence of an enzyme capable of converting said prodrug to said serotonin receptor agonist, thereby stimulating said cells, wherein said cells are either autologous cells obtained from said subject or allogeneic cells obtained from a donor; and
(ii) administering a therapeutically effective amount of the stimulated cells obtained in step (i) to said subject to thereby treat said disease, disorder or condition.

34. The method of claim 33, further comprising the steps of removing excess of said serotonin receptor agonist and/or prodrug thereof from said composition; and optionally diluting the composition thus obtained, prior to step (ii).

35. The method of claim 34, wherein said removing excess of said serotonin receptor agonist and/or prodrug thereof from said composition is carried out by washing the stimulated cells obtained in step (i).

36. The method of claim 33, wherein said cells are selected from the group consisting of bone marrow cells, stem cells, lymphocytes, white blood cells, CAR-T cells, CAR-NK cells, and natural killer cells.

37. The method of claim 33, wherein said serotonin receptor is selected from the group consisting of 5-HT1A, 5-HT1B, 5-HT1E, 5-HT2A, 5-HT2B, 5-HT2C, 5-HT3A, 5-HT3, 5-HT4, and 5-HT7 receptors, and a combination thereof.

38. The method of claim 33, wherein said serotonin receptor agonist is selected from the group consisting of a tryptamine, phenethylamine, ergoline, and a derivative, analog, or salt thereof; and/or said enzyme is a phosphatase selected from the group consisting of an alkaline phosphatase, esterase, and hydrolase.

39. The method of claim 38, wherein said serotonin receptor agonist is selected from the group consisting of urapidil, 5-methyl-urapidil, quipazine, lysergic acid diethylamide (LSD), 1-(2,5-dimethoxy-4-methylphenyl)-2-aminopropane, aripiprazole, sumatriptan, CGS 12066B, CP-94,253, flesinoxan, mirtazapine, m-chlorophenylpiperazine, norfenfluramine, ergotamine, methylergonovine, liseride, fenfluramine, dihydroergotamine, pergolide, cabergoline, terguride, piribedil, bufotenine, 2-methyl-5-HT, phenylbiguanide, 2,5-dimethoxy-4-iodoamphetamine, 3,4-methylenedioxy-methamphetamine, fluoxetine, 5-carboxamidotryptamine, 5-methoxytryptamine, 5-methoxy-a-methyltryptamine, N,N-dimethyltryptamine, 4-fluoro-N,N-dimethyltryptamine, 5-methoxy-N,N-dimethyltryptamine, N,N-diisopropyltryptamine, 4-hydroxy-N,N-diisopropyltryptamine, 4-hydroxy-N-methyl-N-ethyltryptamine, 5-methoxy-N-methyl-N-isopropyl tryptamine, 5-methoxydiisopropyltryptamine, α-methylserotonin, tandospirone, psilocin, 1-methylpsilocin, N-butylpsilocin, 8-hydroxy-2-(di-n-propylamino)tetralin, BW723C86, 4-(4-[4-(2-pyrimidinyl)piperazin-1-yl]butyl)-2,3,4,5-tetrahydro-1,4-benzoxazepine-3,5-dione, gepirone, ipsapirone, tandospirone, N-benzylated analogues of 2,5-dimethoxy-4-iodophenethylamine, buspirone, (+)-cis-8-hydroxy-1-methyl-2-(di-n-propylamino)tetralin, brexpiprazole, and a combination thereof; and said prodrug of said serotonin receptor agonist is selected from the group consisting of psilocybin, N-methylpsilocybin, aripiprazole lauroxil, 1-acetyl-LSD, 1-propionyl-LSD, and 1-butyryl-LSD.

40. The method of claim 39, wherein said cells are treated in step (i) with pergolide; 8-hydroxy-2-(di-n-propylamino)tetralin; or psilocybin, in the presence of alkaline phosphatase capable of hydrolyzing psilocybin to psilocin.

41. The method of claim 33, wherein step (i) is carried out by exposing said cells to said serotonin receptor agonist or prodrug thereof at a concentration of from about 1 μM to about 1 mM.

42. The method of claim 33, wherein said disease, disorder or condition is an immune-related disease, disorder or condition; a cardio-related disease, disorder or condition; a hyperproliferative disorder; or cancer.

43. The method of claim 42, wherein said an immune-related disease, disorder or condition is rheumatoid arthritis, osteoporosis, inflammatory bowel disease, ulcerative colitis, Crohn's disease, malaria, or trypanosomiasis.

44. The method of claim 42, wherein:
(i) said cardio-related disease, disorder or condition is coronary heart disease, chronic heart failure, myocardial infarction, or stroke;
(ii) said hyperproliferative disorder or said cancer is present in the lung, thyroid, head or neck, nasopharynx, throat, nose or sinuses, brain, spine, breast, adrenal gland, pituitary gland, thyroid, lymph, gastrointestinal, mouth, esophagus, stomach, duodenum, ileum, jejunum, small intestine, colon, rectum, genito-urinary tract, uterus, ovary, cervix, endometrial, bladder, testicle, prostate, kidney, pancreas, liver, bone, bone marrow, lymph, blood, skin, or muscle; or
(iii) said cancer is a primary solid cancer such as melanoma, renal cell carcinoma, colon cancer, breast cancer, lung cancer, prostate cancer, bladder cancer, brain cancer, adenocarcinoma of the pancreas, and head and neck tumor, or a metastasis thereof; or a hematological malignancy such as leukemia and lymphoma.

45. An in vitro method for stimulating serotonin receptor-expressing cells to be used either as a therapeutic product or in making a therapeutic product, said method comprising contacting said cells with (a) a serotonin receptor agonist; or (b) a prodrug of said serotonin receptor agonist in the presence of an enzyme capable of converting said prodrug to said serotonin receptor agonist.

46. The method of claim 45, further comprising the steps of removing excess of said serotonin receptor agonist and/or prodrug thereof from said composition; and optionally diluting the composition thus obtained.

47. The method of claim 45, wherein said cells are selected from the group consisting of bone marrow cells, stem cells, lymphocytes, white blood cells, CAR-T cells, CAR-NK cells, and natural killer cells.

48. The method of claim 45, wherein said serotonin receptor is selected from the group consisting of 5-HT1A, 5-HT1B, 5-HT1E, 5-HT2A, 5-HT2B, 5-HT2C, 5-HT3A, 5-HT3, 5-HT4, and 5-HT7 receptors, and a combination thereof.

49. The method of claim 45, wherein said serotonin receptor agonist is selected from the group consisting of a tryptamine, phenethylamine, ergoline, and a derivative, analog, or salt thereof; and/or said enzyme is a phosphatase selected from the group consisting of an alkaline phosphatase, esterase, and hydrolase.

50. The method of claim 49, wherein said serotonin receptor agonist is selected from the group consisting of urapidil, 5-methyl-urapidil, quipazine, lysergic acid diethylamide (LSD), 1-(2,5-dimethoxy-4-methylphenyl)-2-aminopropane, aripiprazole, sumatriptan, CGS 12066B, CP-94,253, flesinoxan, mirtazapine, m-chlorophenylpiperazine, norfenfluramine, ergotamine, methylergonovine, liseride, fenfluramine, dihydroergotamine, pergolide, cabergoline, terguride, piribedil, bufotenine, 2-methyl-5-HT, phenylbiguanide, 2,5-dimethoxy-4-iodoamphetamine, 3,4-methylenedioxy-methamphetamine, fluoxetine, 5-carboxamidotryptamine, 5-methoxytryptamine, 5-methoxy-a-methyltryptamine, N,N-dimethyltryptamine, 4-fluoro-N,N-dimethyltryptamine, 5-methoxy-N,N-dimethyltryptamine, N,N-diisopropyltryptamine, 4-hydroxy-N,N-diisopropyltryptamine, 4-hydroxy-N-methyl-N-ethyltryptamine, 5-methoxy-N-methyl-N-isopropyl tryptamine, 5-methoxydiisopropyltryptamine, a-methylserotonin, tandospirone, psilocin, 1-methylpsilocin, N-butylpsilocin, 8-hydroxy-2-(di-n-propylamino)tetralin, BW723C86, 4-(4-[4-(2-pyrimidinyl)piperazin-1-yl]butyl)-2,3,4,5-tetrahydro-1,4-benzoxazepine-3,5-dione, gepirone, ipsapirone, tandospirone, N-benzylated analogues of 2,5-dimethoxy-4-iodophenethylamine, buspirone, (+)-cis-8-hydroxy-1-methyl-2-(di-n-propylamino)tetralin, brexpiprazole, and a combination thereof; and said prodrug of said serotonin receptor agonist is selected from the group consisting of psilocybin, N-methylpsilocybin, aripiprazole lauroxil, 1-acetyl-LSD, 1-propionyl-LSD, and 1-butyryl-LSD.

51. The method of claim 50, wherein said cells are contacted with pergolide; 8-hydroxy-2-(di-n-propylamino)tetralin; or psilocybin, in the presence of alkaline phosphatase capable of hydrolyzing psilocybin to psilocin.

52. The method of claim 45, wherein said cells are contacted with said serotonin receptor agonist or prodrug thereof at a concentration of from about 1 μM to about 1 mM.
